(19) United States
(12) Patent Application Publication
(10) Pub. No.: US 2024/0033215 A1
(43) Pub. Date: Feb. 1, 2024

(54) INJECTABLE FORMULATION WITH ENHANCED STABILITY CONTAINING DEXIBUPROFEN AND ARGININ AND THEIR MEDICINE VESSEL FOR INJECTION

(71) Applicant: AMOS PHARM, Daejeon (KR)

(72) Inventor: Jae Ho CHOI, Daejeon (KR)

(73) Assignee: AMOS PHARM, Daejeon (KR)

(21) Appl. No.: 18/358,518

(22) Filed: Jul. 25, 2023

(30) Foreign Application Priority Data
Jul. 27, 2022 (KR) 10-2022-0093423

(51) Int. Cl. A61K 9/00; A61K 31/192; A61K 31/198

(52) U.S. Cl. CPC A61K 9/0019; A61K 31/192; A61K 31/198

(57) ABSTRACT

Disclosed herein are an injectable formulation with improved stability and an injectable formulation with improved storage stability. The injectable formulation with improved stability comprises dexibuprofen and arginine and ranging in pH from 6.5 to 8.5, wherein arginine is contained in an amount of 300 to 500 parts by weight and preferably in an amount of 320 to 330 parts by weight, based on 400 parts by weight of dexibuprofen, and the injectable formulation with improved storage stability is prepared by subjecting the injectable formulation with improved stability to nitrogen substitution and loading the nitrogen-substituted formulation into a storage container, with a saturation oxygen of 5% or less therein.

### CROSS-REFERENCE TO RELATED APPLICATION(S)

This application is based on and claims priority under 35 U.S.C. 119 to Korean Patent Application No. 10-2022-0093423, filed on Jul. 27, 2022, in the Korean Intellectual Property Office, the disclosure of which is herein incorporated by reference in its entirety.

### BACKGROUND OF THE INVENTION

### 1. Field of the Invention

The present disclosure relates to an injectable formulation containing dexibuprofen and arginine, with improved stability, and an injection container containing same, with improved storage stability.

### 2. Description of the Prior Art

Ibuprofen is a nonsteroidal anti-inflammatory drug (NSAID) that is used to relieve pain, fever, and inflammation. Ibuprofen has the IUPAC name of 2-(4-isobutylphenyl)propanoic acid with a molecular weight of 206.28, and is available under a number of trade names, including Motrin, Advil, and Nuprin.

Ibuprofen exists as two enantiomers, that is, (R)-ibuprofen and (S)-ibuprofen, with pharmaceutical activity imparted by the S isomer. For ibuprofen formulations sold in pharmacies, they are a racemic mixture. In the body, the (R)-ibuprofen is generally converted into (S)-enantiomer that is active in vivo. The eutomer (S)-ibuprofen is particularly called dexibuprofen.

Dexibuprofen ((S)-ibuprofen) has many advantages over other pain relievers, such as aspirin and acetaminophen, but since dexibuprofen is very poorly soluble in water, many difficulties are encountered in formulating it. Efforts have been made to develop pharmaceutically stable liquid compositions by improving the water solubility.

Dexibuprofen is advantageous over other pain relievers such as aspirin and acetaminophen in terms of various aspects, but has very poor water solubility. Dexibuprofen is a monoprotic acid with pKa=4.4. Its solubility is closely related to pH, varying from 78 μg/mL at acidic pH to 291 mg/mL at alkaline pH. Therefore, efforts are being made to develop specific dosage forms of dexibuprofen, especially injectable formulations, and to improve their stability.

Korean Patent No. 10-2020-0111138 A (Sep. 28, 2020) pertains to a transparent syrup formulation containing dexibuprofen or a pharmaceutically acceptable salt thereof and a solubilizing agent, and a preparation method therefor, wherein the dexibuprofen or the pharmaceutically acceptable salt thereof are in a transparent state and the formulation has a pH of 6 to 8. This document discloses that the syrup formulation is not only highly soluble, but also has pharmacologically active ingredients in a solution state, thus showing excellent bioavailability, and that the formulation does not undergo recrystallization, precipitation, or layer separation even when stored for a long time, exhibiting an excellent effect of improving the homogeneity of the drug effect.

Korean Patent No. 10-2021-0073905 A (Jun. 21, 2021) discloses an injectable formulation containing dexibuprofen and arginine with improved stability, wherein arginine is used in an amount of 344 to 500 parts by weight, based on 400 parts by weight of dexibuprofen 400 and the formulation ranges in pH from 6.5 to 8.5.

Arginine, which is an essential amino acid, serves as a factor essential for converting ammonia to urea in vivo, playing a critical role in cell division, wound healing, removing ammonia from the body, immune function, and the release of hormones. It is a precursor for the synthesis of nitric oxide (NO), making it important in the regulation of blood pressure. Arginine is necessary for T cells to function in the body and can lead to their deregulation if depleted. Arginine is recognized as safe at oral intakes of up to 20 g per day (https://en.wikipedia.org/wiki/Arginine).

However, when injected via an intravenous route, arginine is reported to cause some unwanted effects. Representative of sides effects caused by overdose of intravenous arginine injection are confusion, deep or fast breathing with dizziness, drowsiness, muscle tremors, numbness of the feet, hands, and around the mouth, rapid and deep breathing, restlessness, and stomach cramps (https://www.drugs.com/cons/arginine.html; https://www.mayoclinic.org/drugs-supplements/arginine-intravenous-route/side-effects/drg-20406819?p=1)

Therefore, it is very important from the viewpoint of safety as well as stability to prepare dexibuprofen injections by selecting an optimally minimal amount of arginine as a solubilizing agent for dexibuprofen.

Leading to the present disclosure, intensive and thorough research conducted by the present inventors resulted in the finding that an injectable formulation of dexibuprofen containing arginine in an amount of as small as 320 to 330 parts by weight based on 400 parts by weight of dexibuprofen exhibited safety without side effects of arginine and that an injection container for containing the injectable formulation of dexibuprofen can guarantee excellent storage stability for the injectable formulation when manufactured to have a saturated oxygen of 5% or less therein by nitrogen substitution.

### RELATED ART DOCUMENT

### Patent Literature

### Korean Patent No. 10-2020-0111138 A (Sep. 28, 2020)

### Korean Patent No. 10-2021-0073905 (Jun. 21, 2021)

### SUMMARY OF THE INVENTION

The present disclosure aims to provide an injectable formulation including dexibuprofen and arginine and ranging in pH from 6.5 to 8.5, with improved stability, wherein arginine is contained in an amount of 300 to 500 parts by weight, based on 400 parts by weight of dexibuprofen, and a storage container containing same therein, with improved storage stability.

However, the technical problem to be solved by the present invention is not limited to the above-mentioned problems, and other matters not mentioned can be clearly understood by those skilled in the art from the following description.

The present disclosure provides an injectable formulation including dexibuprofen and arginine and ranging in pH from 6.5 to 8.5, with improved stability, wherein arginine is contained in an amount of 300 to 500 parts by weight and preferably in an amount of 320 to 330 parts by weight, based on 400 parts by weight of dexibuprofen.

The arginine may be L-arginine or D-arginine and the injectable formulation may further include sodium chloride or glucose as an isotonic agent.

The injectable formulation may have a pH of 6.5 to 8.5, an osmotic pressure of 0.1 osmol/kg to 1.0 osmol/kg, and a specific gravity of 1.0 g/mL to 2.0 g/mL.

An aspect of the present disclosure provides a method for preparing an injectable formulation having improved stability, the method comprising a step of adding arginine and dexibuprofen sequentially to water for injection, wherein the arginine is used in an amount of 300 to 500 parts by weight, based on 400 parts by weight of the dexibuprofen.

In addition, an injection container (vial, ampoule, etc.) for containing the injectable formulation of dexibuprofen is manufactured to have a saturated oxygen of 5% or less therein by nitrogen substitution, thus guaranteeing excellent storage stability for the dexibuprofen.

The injectable formulation including dexibuprofen and arginine with improved stability according to the present disclosure contains arginine in an amount of 300 to 500 parts by weight (preferably in an amount of 320 to 330 parts by weight), based on 400 parts by weight of dexibuprofen and has a pH of 6.5 to 8.5, whereby the main component does not precipitate due to the high content of arginine and through pH adjustment. In addition, the injectable formulation has the advantage of exhibiting excellent stability under an accelerated condition.

Moreover, an injection container (vial, ampoule, etc.) for containing the injectable formulation of dexibuprofen is manufactured to have a saturated oxygen of 5% or less therein by nitrogen substitution, thus guaranteeing excellent storage stability for the dexibuprofen.

### BRIEF DESCRIPTION OF THE DRAWINGS

The above and other aspects, features and advantages of the present disclosure will be more apparent from the following detailed description taken in conjunction with the accompanying drawings, in which:

FIG. 1 is a photographic image showing the stability (precipitation) of dexibuprofen in injectable formulations of Examples and Comparative Examples; and

FIG. 2 is a photographic image showing storage stability (color change) of the dexibuprofen injection prepared in Example 1 and loaded and sealed in glass vials against saturated oxygen levels in the storage containers and time (1, 2, 3, and 4 weeks).

### DETAILED DESCRIPTION OF THE EXEMPLARY EMBODIMENTS

Leading to the present disclosure, intensive and thorough research conducted by the present inventors resulted in the finding that when used at a high content in an injectable formulation of dexibuprofen, arginine, available as both a solubilizing agent and a pH adjustor, can enhance the stability of the injectable formulation in an accelerated condition without precipitating the main component and that an injection container for containing the injectable formulation of dexibuprofen can guarantee excellent storage stability for the injectable formulation when manufactured to have a saturated oxygen of 5% or less therein.

Below, a detailed description will be given of the present disclosure.

The present disclosure provides an injectable formulation including dexibuprofen and arginine, with improved stability, wherein arginine is contained in an amount of 300 to 500 parts by weight, based on 400 parts by weight of dexibuprofen and the formulation ranges in pH from 6.5 to 8.5.

First, the injectable formulation according to the present disclosure contains dexibuprofen ((S)-ibuprofen, C₁₃H₁₈O₂) as a main component. Dexibuprofen is the compound represented by the following Chemical Formula 1:

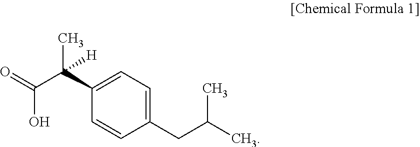

The concentration of dexibuprofen in the injectable preparation may be determined in consideration of usage and dosage and may range from 0.1 mg/mL to 10 mg/mL and preferably from 0.4 mg/mL to 4 mg/mL, but is not limited thereto.

Next, the injectable formulation according to the present disclosure contains arginine that serves both to dissolve dexibuprofen, poorly soluble in water, and to adjust the pH. The arginine may be L-arginine or D-arginine.

In the present disclosure, arginine is contained in an amount of 300 to 500 parts by weight and preferably in an amount of 320 to 330 parts by weight, based on 400 parts by weight of dexibuprofen, but with no limitations thereto. With the ability to both smoothly dissolve dexibuprofen and adjust pH, such a high content of arginine is advantageous in imparting excellent stability to the injectable formulation in an accelerated condition. A higher content of arginine can further improve the stability of the injectable formulation in an accelerated condition.

However, given an arginine content exceeding 500 parts by weight, the cationicity due to the arginine molecule increases to greatly lower the stability of the injectable formulation, rather. An arginine content below 320 parts by weight cannot sufficiently dissolve dexibuprofen, thus decreasing the stability of the injectable formulation.

In order to set the concentrations of dexibuprofen and arginine in the injectable formulation according to the present disclosure as described in the foregoing, they need to be diluted in water for injection. In this regard, the water for injection may further include sodium chloride or glucose as an isotonic agent. In detail, 0.1-2.0% physiological saline injection, 1-10% glucose injection, or lactated Ringer's solution may be used as water for injection.

In addition, the injectable formulation according to the present disclosure may have a pH of 6.5 to 8.5 and preferably a pH of 6.5 to 7.5, but with no limitations thereto. In order to adjust the pH, hydrochloric acid and the like may be contained. A pH less than 6.5 may cause the deposition of the main component (that is, a precipitate occurs). At a pH of higher than 8.5, the injectable formulation is likely to allow microbial generation and growth.

The injectable formulation according to the present disclosure may have an osmotic pressure of 0.1-1.0 osmol/kg.

The injectable formulation according to the present disclosure may have a specific gravity of 1.0-2.0 g/mL.

Moreover, the injectable formulation according to the present invention has the advantage of being superb in terms of stability without precipitating the main component even in an accelerated condition and an injection container for containing the injectable formulation is manufactured to have a saturated oxygen of 5% or less therein by nitrogen substitution, thus guaranteeing excellent storage stability for the dexibuprofen.

In addition, the present disclosure provides a method for preparing an injectable formulation having improved stability, the method including a step of adding arginine and dexibuprofen sequentially to water for injection, wherein the arginine is used in an amount of 300 to 500 parts by weight, based on 400 parts by weight of dexibuprofen and the formulation has a pH of 6.5 to 8.5.

As stated in the foregoing, the injectable formulation with improved stability according to the present disclosure, characterized by containing arginine at a content of 300 to 500 parts by weight (preferably 320 to 330 parts by weight) based on 400 parts by weight of dexibuprofen and having a pH of 6.5 to 8.5, does not undergo the precipitation of the main component due to the high content of arginine and pH adjustment, and a container for containing the injectable formulation therein is manufactured to have a saturated oxygen of 5% or less through nitrogen substitution, thus guaranteeing excellent storage stability for dexibuprofen.

Also contemplated according to the present disclosure are an injection containing dexibuprofen and arginine and an injection container containing same therein.

The method for preparing the injectable formulation includes the steps of:
dissolving an appropriate amount of arginine in a nitrogen-substituted water for injection at 30° C. or less while stirring (process 1);
stirring an appropriate amount of dexibuprofen at 25˜30° C. in the solvent of process 1 while maintaining the solution at 25-30° C. (process 2);
completely dissolving the solution of process 2 by adjusting the pH of the solution into 7.5±0.2 with HCl (process 3);
making a predetermined concentration by introducing an additional water for injection to the solution of process 3 (process 4);
sterilizing and filtering the solution of process 4 by passage through a 0.2-μg filter (process 5);
filling an injection container with the solution of process 5 (process 6);
passing the filled storage container of 6 through a nitrogen tunnel to reduce the saturation oxygen therein to 5% or less by nitrogen substitution and then sealing the container (process 7); and
autoclaving the injection-contained storage container of process 7 at 121° C. for 20 minutes (process 8).

A better understanding of the present disclosure may be obtained through the following examples, which are set forth to illustrate, but are not to be construed to limit the present disclosure.

### EXAMPLES

### Examples 1-4 and Comparative Examples 1-4: Preparation of Injectable Formulations

Injectable formulations were prepared to have the compositions of Table 1, below. Briefly, L-arginine and dexibuprofen were sequentially added to water for injection, followed by sodium chloride or glucose as an isotonic agent. After dissolution of the ingredients, hydrochloric acid was added as a pH adjustor to adjust the pH. As a result, injectable formulations of dexibuprofen with a final volume of 100 mL were prepared.

### Preparation of Example 1

To 80 mL of nitrogen-substituted water for injection was added 320 mg of arginine, followed by dissolution through stirring at 30° C. or less for 30 minutes. While the temperature was maintained at 25˜30° C., the solution was stirred, together with 400 mg of dexibuprofen, at 25-30° C. for 60 minutes. The mixture was completely dissolved by adjusting the pH into 7.5±0.2 with HCl. Water for injection was additionally added to form a final volume of 100 mL. The solution was sterilized and filtered through a 0.2-μm filter and autoclaved at 121° C. for 20 minutes to afford an injectable formulation.

TABLE 1

| | | | | C. | C. | C. | C. |
|---|---|---|---|---|---|---|---|
| Ingredient | Ex. 1 | Ex. 2 | Ex. 3 | Ex. 1 | Ex. 2 | Ex. 3 | Ex. 4 |
| Dexibuprofen | 400 | 400 | 400 | 400 | 400 | 400 | 400 |
| (mg) | | | | | | | |
| L-arginine (mg) | 320 | 350 | 400 | 100 | 150 | 280 | 320 |
| NaCl (mg) | 900 | 900 | — | 900 | 900 | 900 | 900 |
| Glucose (mg) | — | — | 5000 | — | — | — | — |
| pH Adjustor | suitable | suitable | suitable | suitable | suitable | suitable | suitable |
| Water for | suitable | suitable | suitable | suitable | suitable | suitable | suitable |
| injection (ml) | | | | | | | |
| pH | 7.5 | 7.5 | 7.5 | 7.5 | 7.5 | 7.5 | 5.5 |

### [Experimental Example 1] Assay for Precipitation in Injectable Formulation of Dexibuprofen

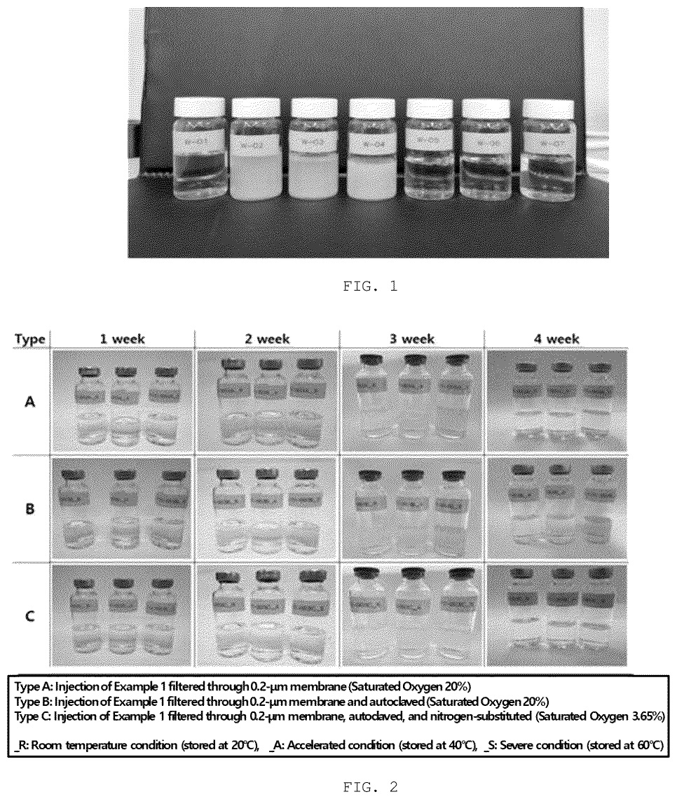

It was examined with the naked eye whether or not precipitation occurred within 24 hours in the injectable formulations of dexibuprofen prepared in Examples 1 to 3 and Comparative Examples 1 to 4, and the results are shown in FIG. 1.

As shown in FIG. 1, it was observed that precipitates of the main component were formed in the injectable formulations of dexibuprofen prepared in Comparative Examples 1 to 4 whereas the injectable formulations of dexibuprofen prepared in Examples 1 to 3 did not allow the main component to precipitate and thus remained transparent.

### [Experimental Example 2] Measurement of Oxygen Level in Injection Container

As for the oxygen levels in the injection containers containing dexibuprofen therein according to the present disclosure, saturated oxygen (%) was measured using the analyzer Gaspace Advance from SYSTECH Illinois.

### [Experimental Example 3] Assay for Stability of Dexibuprofen Injection

The dexibuprofen injection prepared in Example 1 was loaded into glass vials and sealed to give samples which were then examined for storage stability according to saturated oxygen levels in the injection storage containers. In this regard, before being loaded into storage containers, the injectable formulation of Example 1 was filtered through a 0.2-μm filter for Type A, filtered through a 0.2-μm filter and then autoclaved for Type B, and filtered through a 0.2-μm filter, subjected to nitrogen substitution, and then autoclaved for Type C. Saturated oxygen in the storage containers was measured to be 20% for Type A, 20% for Type B, and 3.65% for Type C.

The stability of each sample was evaluated for 1 to 4 weeks in a stability chamber under accelerated conditions (60° C., 40° C., and room temperature and 75% relative humidity). Briefly, after storage for 1 to 4 weeks, the contents of dexibuprofen relative to the initial amount (input) thereof were analyzed by high-performance liquid chromatography and color changes (colorless→yellowish white→pale yellow→yellow) were monitored. The results are summarized in Tables 2, 3, and 4 and depicted in FIG. 2.

TABLE 2
Stability Test in 60° C. condition

| | | | Test Duration | | | | |
|---|---|---|---|---|---|---|---|
| Type | Test item | Test Standard | Initial | Week 1 | Week 2 | Week 3 | Week 4 |
| A | Appearance | Colorless or pale- | Colorless | Yellowish | Pale | Pale | Yellow |
| | | yellow transparent | | white | yellow | yellow | |
| | | liquid | | | | | |
| | pH | pH 6.5~pH 8.5 | 7.62 | 7.61 | 7.53 | 7.56 | 7.62 |
| B | Appearance | Colorless or pale- | Colorless | Yellowish | Yellowish | Pale | Yellow |
| | | yellow transparent | | white | white | yellow | |
| | | liquid | | | | | |
| | pH | pH 6.5~pH 8.5 | 7.63 | 7.56 | 7.51 | 7.56 | 7.57 |
| C | Appearance | Colorless or pale- | Colorless | Colorless | Colorless | Colorless | Yellowish |
| | | yellow transparent | | | | | white |
| | | liquid | | | | | |
| | pH | pH 6.5~pH 8.5 | 7.65 | 7.65 | 7.57 | 7.66 | 7.73 |

TABLE 3
Stability Test in 40° C. condition

| | | | Test Duration | | | | |
|---|---|---|---|---|---|---|---|
| Type | Test item | Test Standard | Initial | Week 1 | Week 2 | Week 3 | Week 4 |
| A | Appearance | Colorless or pale- | Colorless | Colorless | Yellowish | Pale | Pale |
| | | yellow transparent | | | white | yellow | yellow |
| | | liquid | | | | | |
| | pH | pH 6.5~pH 8.5 | 7.62 | 7.61 | 7.53 | 7.56 | 7.62 |
| B | Appearance | Colorless or pale- | Colorless | Colorless | Colorless | Yellowish | Pale |
| | | yellow transparent | | | | white | yellow |
| | | liquid | | | | | |
| | pH | pH 6.5~pH 8.5 | 7.63 | 7.56 | 7.51 | 7.56 | 7.57 |
| C | Appearance | Colorless or pale- | Colorless | Colorless | Colorless | Colorless | Colorless |
| | | yellow transparent | | | | | |
| | | liquid | | | | | |
| | pH | pH 6.5~pH 8.5 | 7.65 | 7.65 | 7.57 | 7.66 | 7.73 |

TABLE 4
Stability Test in 20° C. condition

| | | | Test Duration | | | | |
|---|---|---|---|---|---|---|---|
| Type | Test item | Test Standard | Initial | Week 1 | Week 2 | Week 3 | Week 4 |
| A | Appearance | Colorless or pale- | Colorless | Colorless | Colorless | Colorless | Colorless |
| | | yellow transparent | | | | | |
| | | liquid | | | | | |
| | pH | pH 6.5~pH 8.5 | 7.62 | 7.62 | 7.66 | 7.68 | 7.74 |
| | Content | 90.0~110.0% | 100.80 | | 99.20 | | 98.20 |
| B | Appearance | Colorless or pale- | Colorless | Colorless | Colorless | Colorless | Colorless |
| | | yellow transparent | | | | | |
| | | liquid | | | | | |
| | pH | pH 6.5~pH 8.5 | 7.63 | 7.63 | 7.6 | 7.69 | 7.74 |
| | Content | 90.0~110.0% | 100.80 | | 99.60 | | 99.2 |
| C | Appearance | Colorless or pale- | Colorless | Colorless | Colorless | Colorless | Colorless |
| | | yellow transparent | | | | | |
| | | liquid | | | | | |
| | pH | pH 6.5~pH 8.5 | 7.65 | 7.65 | 7.61 | 7.69 | 7.73 |
| | Content | 90.0~110.0% | 100.80 | | 100.20 | | 100.00 |

What is claimed is:

1. An injectable formulation, comprising dexibuprofen and arginine and ranging in pH from 6.5 to 8.5, with improved stability,
wherein arginine is contained in an amount of 320 to 330 parts by weight, based on 400 parts by weight of dexibuprofen.

2. The injectable formulation of claim 1, wherein the arginine is L-arginine or D-arginine

3. The injectable formulation of claim 1, further comprising sodium chloride or glucose as an isotonic agent.

4. The injectable formulation of claim 1, wherein the injectable formulation has an osmotic pressure of 0.1 osmol/kg to 1.0 osmol/kg and a specific gravity of 1.0 g/mL to 2.0 g/mL.

5. An injectable formulation with improved storage stability, prepared by subjecting the injectable formulation of claim 1 to nitrogen substitution and loading the nitrogen-substituted formulation into a storage container, with a saturation oxygen of 5% or less therein.

6. A method for preparing an injectable formulation having improved storage stability, the method comprising the steps of:
dissolving an appropriate amount of arginine in a nitrogen-substituted water for injection at 30° C. or lower while stirring to prepare a solution(process 1);
stirring an appropriate amount of dexibuprofen at 25˜30° C. in the solution of process 1 while maintaining the solution at 25-30° C. (process 2);
completely dissolving the solution of process 2 by adjusting the pH of the solution into 7.5±0.2 with HCl (process 3);
making a predetermined concentration by introducing an additional water for injection to the solution of process 3 (process 4);
sterilizing and filtering the solution of process 4 by passage through a 0.2 pm filter (process 5);
filling a storage container for injection with the solution of process 5 (process 6);
passing the filled storage container of 6 through a nitrogen tunnel to reduce the saturation oxygen therein to 5% or less by nitrogen substitution and then sealing the container (process 7); and
autoclaving the storage container containing the injection solution of process 7 at 121° C. for 20 minutes (process 8).

7. An injectable formulation with improved storage stability, prepared by subjecting the injectable formulation of claim 2 to nitrogen substitution and loading the nitrogen-substituted formulation into a storage container, with a saturation oxygen of 5% or less therein.

8. An injectable formulation with improved storage stability, prepared by subjecting the injectable formulation of claim 3 to nitrogen substitution and loading the nitrogen-substituted formulation into a storage container, with a saturation oxygen of 5% or less therein.

9. An injectable formulation with improved storage stability, prepared by subjecting the injectable formulation of claim 4 to nitrogen substitution and loading the nitrogen-substituted formulation into a storage container, with a saturation oxygen of 5% or less therein.
